(19) United States
(12) Patent Application Publication
(10) Pub. No.: US 2023/0280339 A1
(43) Pub. Date: Sep. 7, 2023

(54) PHOTOACOUSTIC ASSAY BASED ON OPTICALLY ACTUATED GOLD NANOPARTICLES FOR THE DETECTION OF BIOLOGICAL ANALYSTS

(71) Applicants: Board of Regents, The University of Texas System, Austin, TX (US); Avsana Labs Incorporated, Richardson, TX (US)

(72) Inventors: Zhenpeng Qin, Austin, TX (US); Dennis I. Robbins, Richardson, TX (US); Nadine va Zyl, Richardson, TX (US); Haihang Ye, Austin, TX (US); Peiyuan Kang, Austin, TX (US)

(21) Appl. No.: 18/176,056

(22) Filed: Feb. 28, 2023

Related U.S. Application Data
(60) Provisional application No. 63/315,361, filed on Mar. 1, 2022.

(51) Int. Cl. G01N 33/543; B01L 3/00; G01N 33/569

(52) U.S. Cl. CPC G01N 33/54373; G01N 33/54346; B01L 3/502761; G01N 33/56983; B01L 2200/16; B01L 2300/0636; B01L 2200/0652

(57) ABSTRACT

A biochemical assay device including a detection channel, a light source and a hydrophone. The detection channel fluidly coupleable to a specimen source to receive an analyte sample that can include a biological target bound to a bioreceptor gold nanoparticle conjugate. The light source situated to send a light to the detection channel, where the light includes one or more wavelengths absorbable by the bioreceptor gold nanoparticle conjugate bound to the biological target to thereby generate a photoacoustic signal indicative of an individual acoustic detection event from the bioreceptor gold nanoparticle conjugates to the biological target in the analyte sample. The hydrophone to detect and convert a summation of the photoacoustic digital signals into an electrical signal.

### CROSS-REFERENCE TO RELATED APPLICATION

This application claims the benefit of U.S. Provisional Application Ser. No. 63/315,361, filed by Zhenpeng Qin, et al. on Mar. 1, 2022, entitled “PHOTOACOUSTIC ASSAY BASED ON OPTICALLY ACTUATED GOLD NANOPARTICLES FOR THE DETECTION OF BIOLOGICAL ANALYSTS,” commonly assigned with this application and incorporated herein by reference in its entirety.

### GOVERNMENT SUPPORT

This invention was made with government support under Grant no. R01 AI151374 awarded by the National Institutes of Health. The government has certain rights in the invention.

### TECHNICAL FIELD

This application is directed, in general, to biochemical assay devices, assay systems and methods for detecting biological targets in diagnostic or environmental specimens or samples, and more specifically, viruses, bacteria, pathogens, proteins, metabolites, molecules, DNA, RNA, or other biological targets materials using the biochemical assay device.

### BACKGROUND

Medical diagnostics is a key element of healthcare infrastructure. Due to the necessity of providing practitioners with diagnostic information in order for them to prescribe appropriate therapeutics and treatment, diagnostics are often a bottleneck in patient care. Diagnostic analyses often require highly specialized equipment that is both expensive and requires highly trained operators. Frequently, diagnostic samples must be sent to a lab facility that is not at point-of-care (PoC), resulting in delays in analysis. In addition, for the practitioner to prescribe therapy and a treatment regimen, as well as be cognizant of contagion risks, it is valuable to have the capability to test for multiple virus types simultaneously, in a multiplexed manner. There is a need for a method and apparatus of carrying out digital photoacoustic-based and microbubble (MB)-enhanced photoacoustic (PA) effect-based virus detection and quantification on prepared biological samples, and preferably in a multiplexed manner. There is a need for a user-friendly device that can deliver a diagnostic result on several samples in a short time.

As an example, The COVID-19 pandemic has had a substantial impact on global society. Among the changes is an increased need for PoC testing that is rapid, sensitive, specific, and low-cost. Also, to help limit viral spread, environmental virus monitors have been sought for use in medium to high traffic public locations; however, the effectiveness of current monitors is limited by the sensitivity of the applied device, especially in regard to dilute media. As of May 2021, over 160 million confirmed cases and over 3.4 million deaths have been attributed to the ongoing worldwide outbreak of novel coronavirus SARS-CoV-2. It most commonly manifests as an acute illness with symptoms including fever, cough, myalgia, and fatigue. As these symptoms are similar to those caused by influenza viruses, it is often difficult to distinguish between the viruses. Moreover, both can simultaneously co-infect a patient. Likewise, the Zika virus usually co-circulates with Dengue and Chikungunya viruses in the same geographical areas during the same seasonal period through the same biting arthropods. Such examples motivate the need for a general method that can distinguish different pathogens and provide a reliable, early, and accurate multiplexed diagnosis that is crucial to providing timely medical treatment for patients, as well as mitigating the spread of disease.

Digital assays, a technological breakthrough in testing, significantly enhance sensitivity by allowing for the separation of analytes into sub-nanoscale partitions, giving a 1 or 0 signal for the presence or absence of a single molecule of interest. However, current digital assays require large-scale, specialized instruments to perform multiple sample preparation and analysis steps, as well as rely on a large number of microscale partitions (10⁴˜10⁷, microwells or droplets) and fluorescent detection or imaging. It remains a major scientific challenge to develop, validate, and apply low cost, rapid and ultrasensitive digital assays for infectious diseases.

Current diagnostic tests are composed of two main categories: nucleic acid tests and protein/antigen tests. Polymerase chain reaction (PCR), a nucleic acid test and the current “gold” standard in diagnostic testing, provides accurate results, but is costly with a number of limitations. Rapid antigen tests, on the other hand, are simple, fast, typically well-suited for use at the PoC, but lack sufficient sensitivity. In order to combat the limitations of current diagnostic approaches, various novel methods have been or are currently in development. Though such methods promise advantages such as rapid turnaround and high sensitivity, each exhibits its own range of limitations. There is a significant business, economic, and societal need for assay that is rapid, highly sensitive, multiplexed for multiple biological target types and analyte samples from multiple sources, and inexpensive using a partition-free digital PA effect-based technology. The approach will also provide a fast response for epidemics that share similar clinical presentations during the same seasonal period. Furthermore, due to its very high sensitivity, the technology will be applicable not only to viral diagnostics, but to environmental virus monitoring, and able to monitor for multiple viral pathogens simultaneously due to its multiplexed capability.

### SUMMARY

The present disclosure provides in one embodiment, a biochemical assay device that includes a detection channel fluidly coupleable to a specimen source to receive an analyte sample that can include a biological target bound to a bioreceptor gold nanoparticle conjugate, a light source situated to send a light to the detection channel, where the light includes one or more wavelengths absorbable by the bioreceptor gold nanoparticle conjugate bound to the biological target to thereby generate a photoacoustic signal indicative of an individual acoustic detection event from the bioreceptor gold nanoparticle conjugates to the biological target in the analyte sample and a hydrophone to detect and convert a summation of the photoacoustic digital signals into an electrical signal

### BRIEF DESCRIPTION OF FIGURES

For a more complete understanding of the present disclosure, reference is now made to the following detailed description taken in conjunction with the accompanying FIGUREs, in which:

FIG. 1 presents a schematic illustration for an assay systems design according to the principles of the present disclosure;

FIG. 2 presents a block diagram of aspects of biochemical assay device embodiments of the present disclosure;

FIG. 3 presents a schematic illustration of aspects of engineering gold nanoparticle (AuNPs) and microbubbled (MBs) for virus binding, and the production of the gold nanoparticle conjugated microbubbles (MB-V-AuNP);

FIGS. 4A-4C presents a schematic illustration of size-based separation of MB-V-AuNPs by a deterministic lateral displacement (DLD) microfluidic device, whereby in: (A), the device uses an array of regularly disposed pillars to orient the fluid path in a microfluidic channel, resulting in a size-based separation trajectory for different particles where the free AuNPs flow into the control channel on the top of the DLD, in (B), the geometry of micropillar structures in the DLD channel creates different trajectories for particles of different sizes. Particles labeled 1, 2, 3 correspond to channels 1, 2, and 3, respectively, and, in C, parameters for the DLD device design are shown for such an example setup;

FIG. 5 presents a schematic illustration of example PA signals from different channels collected by a signal processor and distinguished via time delay, where an example time delay of 10 μs corresponds to a 15 mm interval distance between channel and the difference allows for the distinguishing of different sizes of MBs, and thus, different targets;

FIGS. 6A-6F present a schematic illustration of the photoacoustic detection of a single MB-V-AuNP, whereby: (A) the PA amplitude of a gold nanoparticle coated microbubble (AuMB) is compared with that of free gold nanoparticles (AuNPs), (B) shows the vibration of an AuMB fitted to a theoretical function, (C) shows the PA signal generated from a single AuNP (D) shows a comparison of PA pressure generated from single AuNP, background, control, and single MB-V-AuNP (E) shows the microchannel setup for PA detection, (F) shows PA waveforms for different samples; and

FIG. 7 presents example normalized optical density graph for absorption spectra applicable to various sizes of AuNPs.

### DETAILED DESCRIPTION

As part of the present invention the technology disclosed herein addresses assay systems for detecting analyte targets in samples, particularly from biological samples. In particular, the technology relates to microfluidic systems that carry out viral or protein detection and quantification on viruses, pathogens, proteins, or molecules of interest. Of particular interest are systems that perform multiplexed operation capable of testing analytes for multiple target types, or capable of multiplexed testing of analytes from a plurality of sources in a parallel or serial manner.

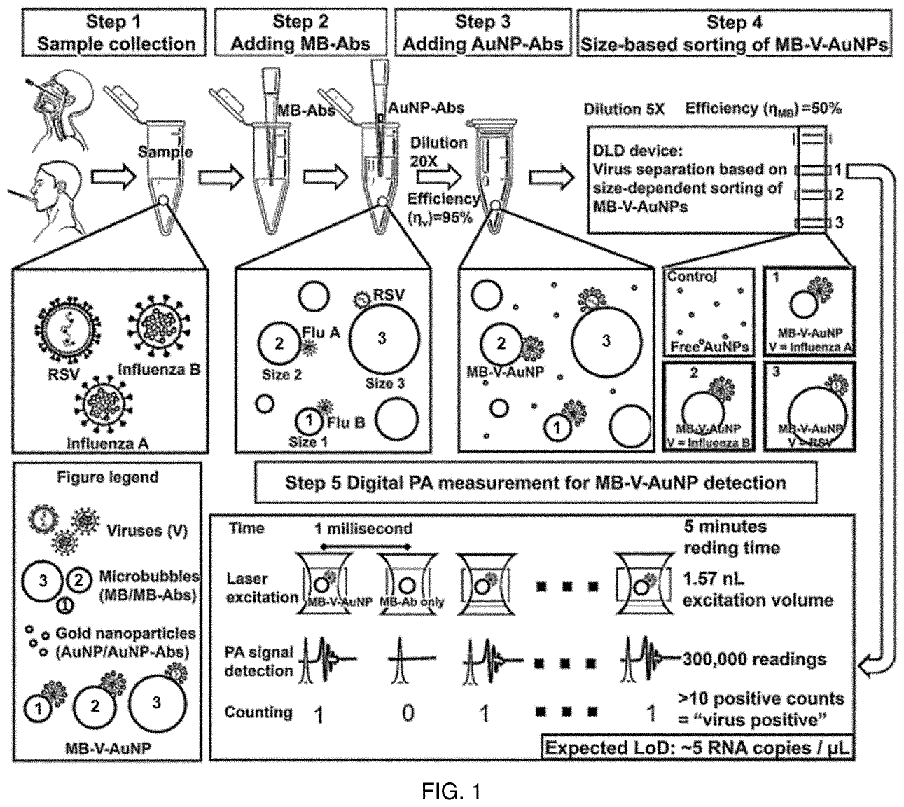

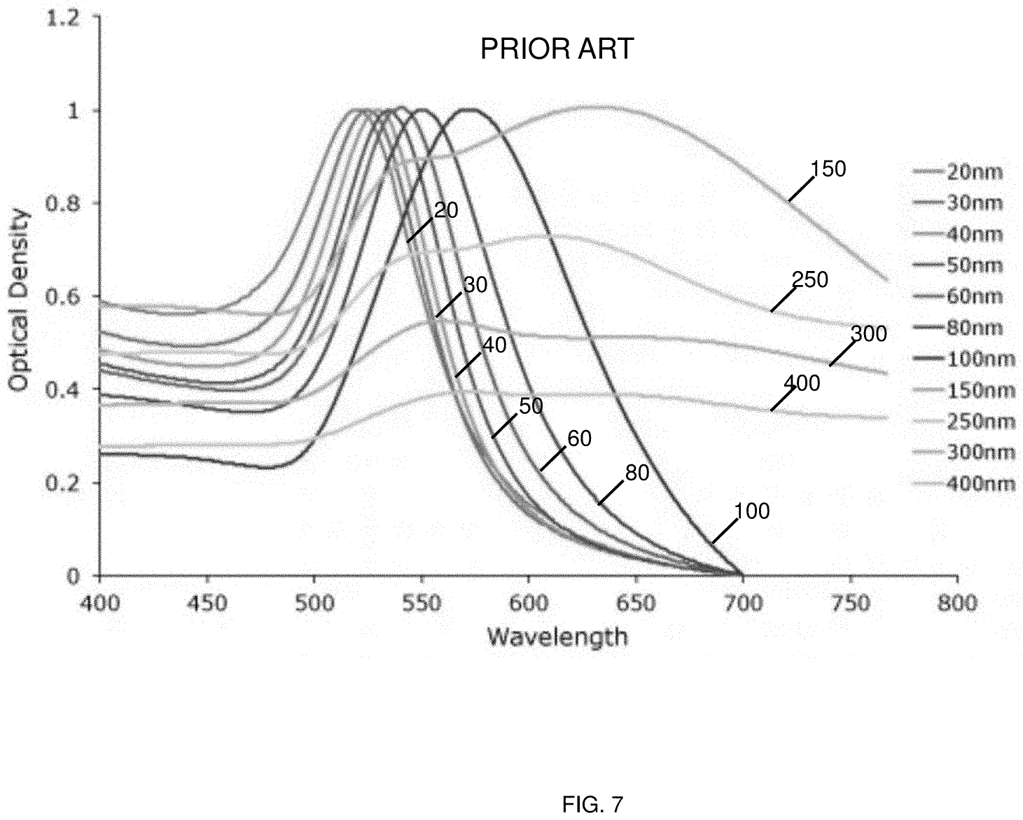

Embodiments of the disclosure include a biochemical assay device based on partition-free digital detection of a photoacoustic (PA) signal employing and resulting from optically actuated gold nanoparticles (AuNPs) when conjugated to appropriate analyte targets is disclosed. Methods of operating the device in a multiplexed manner are disclosed. Embodiments of the device can include one or more pulsed lasers, one or more PA detection channels, one or more ultrasound transducers, one or more signal amplifiers, signal processing functionality, and human interface functionality. Antibody-conjugated or aptamer-conjugated AuNPs as bioreceptors bind to appropriate analyte targets (viruses, proteins or other molecules) enabling photoacoustic detection of the analyte targets. In one embodiment of the device, a microbubble (MB)-enhanced photoacoustic (PA) signal from MBs conjugated to said AuNPs is employed, and a method of operating said device in a multiplexed manner with the use of a deterministic lateral displacement (DLD) device, the DLD provided, with a microfluidic channel containing a plurality of regions in order to separate a plurality of types of particles: free AuNPs and two or more analyte targets (viruses, proteins or other) of interest (FIG. 1, 2, 3). In another embodiment of the device, AuNPs of a plurality of sizes are employed, having optical absorption spectra specific to each size of AuNP, with each size conjugated to a specific type of bioreceptor, such that the spectral response specific to each size of AuNP may be used to differentiate and determine the target type based on spectral response specific to that corresponding size of AuNP (FIG. 7).

For instance, in the case of a multiplexed operation of the PA device, a specific size AuNPs may be conjugated to a bioreceptor specific to a particular analyte target, e.g., antibody specific to RSV virus. In the case of a single pulsed laser of a particular wavelength, the relative amplitude of the acoustic response when an analyte target particle is detected may be used to identity the analyte target type of interest when compared to the amplitude of acoustic response from other target analytes. In the case where multiple pulsed lasers are used to actuate an acoustic signal, the pulses can be spaced close together, e.g., a few microseconds apart, and the acoustic response to the pulse from one laser relative to the acoustic response to the pulse from a second laser of different wavelength may be used to identify spectroscopically the particular analyte target type of interest, such as illustrated for example data presented in FIG. 7 (sourced from Cytodiagnostics Inc.).

Embodiments of the device can include microfluidic systems that perform biological target detection employing PA effect detection technology. Embodiment of the device can perform multiplexed operation capable of testing for multiple analyte types from a single sample or specimen, employing a single instrument or device.

Embodiments of the device can include one or more pulsed lasers, one or more PA detection channels, one or more ultrasound transducers, one or more signal amplifiers, signal processing functionality, and human interface functionality. Antibody-conjugated or aptamer-conjugated AuNPs as bioreceptors bind to appropriate analyte targets (viruses, proteins or other) enabling photoacoustic detection of said analyte targets.

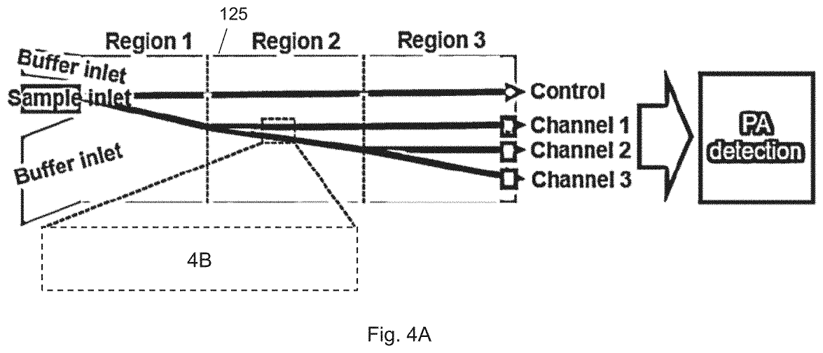

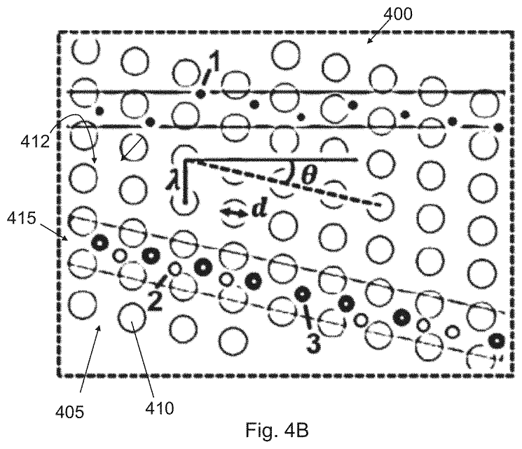

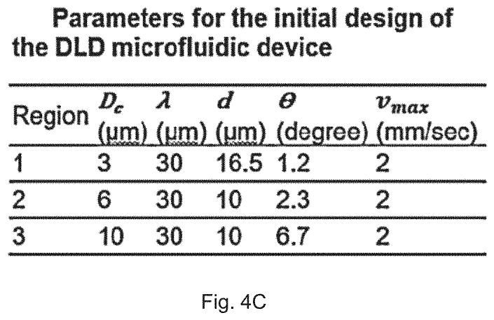

In some embodiments of the device, a microbubble (MB)-enhanced photoacoustic (PA) signal from MBs conjugated to the AuNPs is employed, and a method of operating the device in a multiplexed manner with the use of a deterministic lateral displacement device (DLD, FIG. 4A-4C), is provided, with a microfluidic channel containing a plurality of regions configured to separate a plurality of types of particles: free AuNPs and two or more analyte targets (viruses, proteins or other) of interest (FIG. 1).

For instance, in the schematic illustration presented in FIG. 1, antibody-conjugated MBs (MB-Abs) attach to specified viral targets, which then attach to antibody-conjugated gold nanoparticles (AuNP-Abs). The multiplexed separation can be accomplished by using MB-Abs of different sizes (corresponding to size numbers 1, 2, and 3) to bind with different viruses. MB-Abs that specifically bind with one type of virus can be further separated from other MB-Abs by a microfluidic device based on their size differences. These structures are actuated with laser pulses at wavelengths absorbed by the AuNPs. The energy transferred to the MBs generates an enhanced photoacoustic (PA) signal that can be used to detect and differentiate the target viruses.

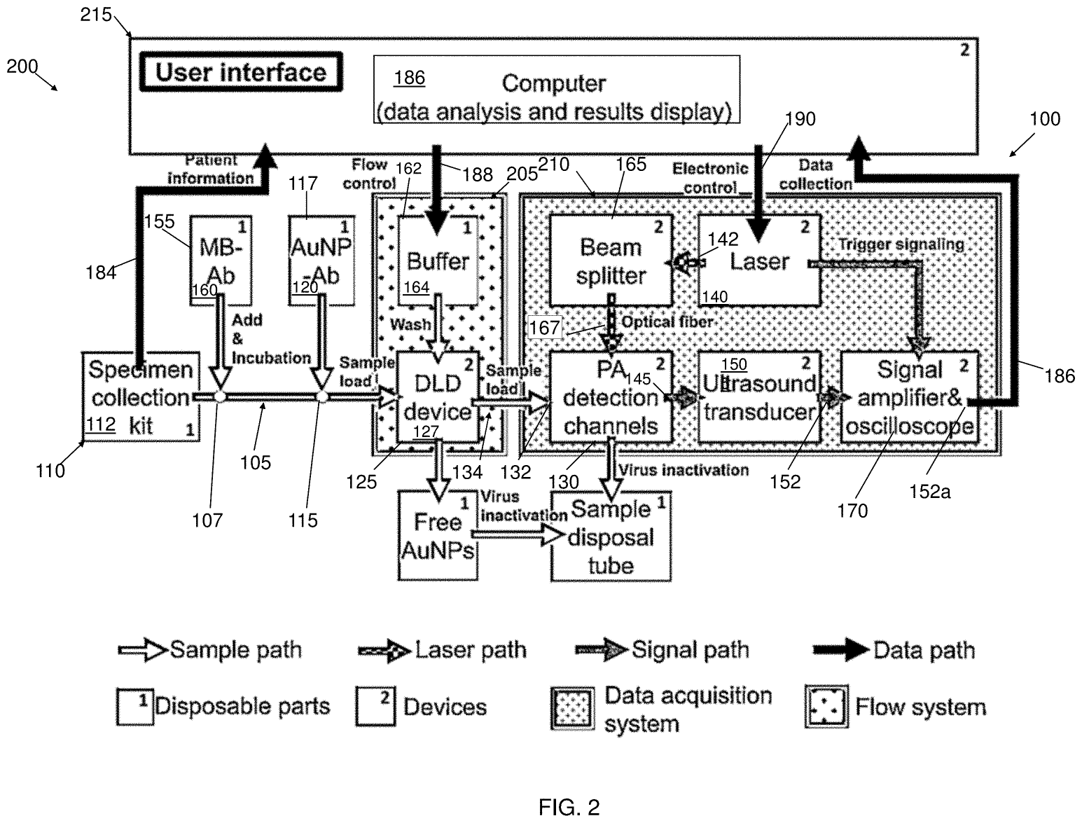

In the block diagram of the device presented in FIG. 2, all disposable parts can be arranged to allow easy access for users. There are three major components: user interface, data acquisition system, and flow system. The flow system can include the DLD device, which facilitated target separation. The data acquisition system can include laser, ultrasound transducer, and signal amplifier devices. A beam splitter can be coupled to the PA detection channels via an optical fiber. During testing, a focused, pulsed laser beam creates an excitation volume, and the PA signal of the MB-V-AuNPs can be measured by a hydrophone. Signal processing functionality (for example, via a signal amplifier and oscilloscope) allows for collection of the PA signal. Additionally, the user interface (UI) can be configured to allow the user to input patient information, and test results can be automatically collected and analyzed by the UI. The UI can allow users to control the flow and data acquisition systems by program. In some device embodiments the DLD device can be optional. In the case of PA signal generation without the use of MBs, the DLD device shown in this diagram can be omitted, and the block labeled “DLD device” instead be a mixing chamber. In some such device embodiments, the block labeled MB-Ab can also be omitted.

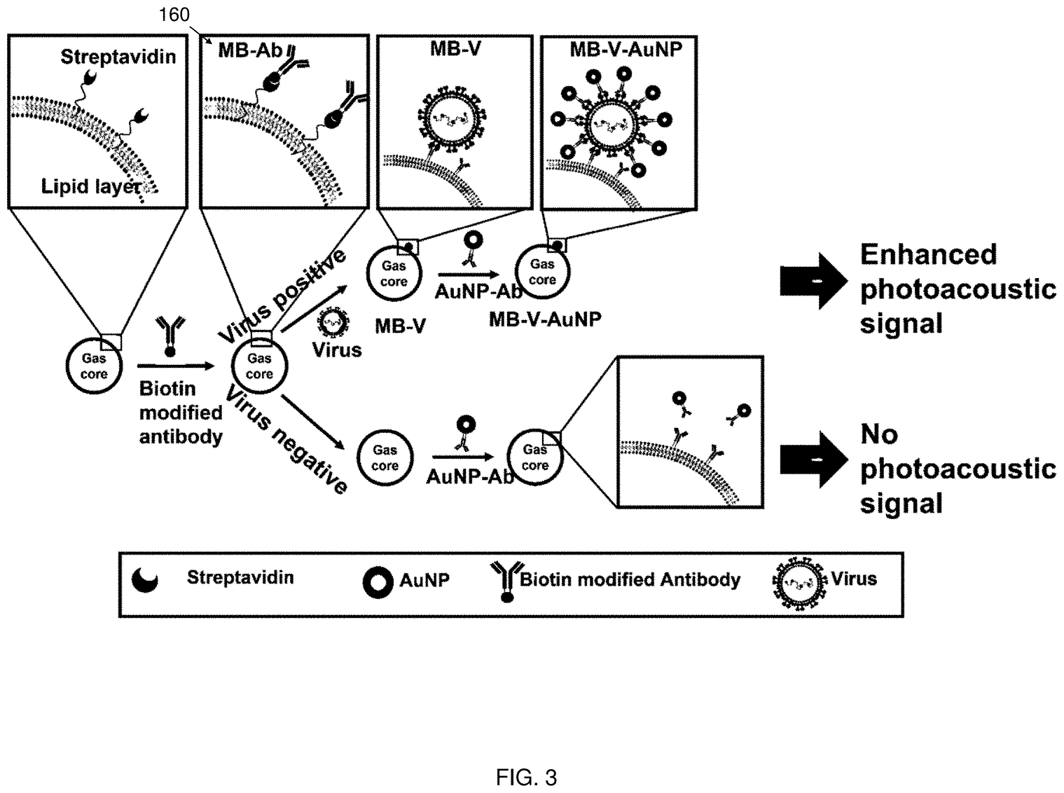

As illustrated in FIG. 3, in some embodiments of the device, antibody-functionalized MBs (MB-Abs) are prepared by linking streptavidin modified MBs with biotin-modified antibodies that recognize a specified protein on the viral target. AuNPs can be prepared and modified with antibodies that recognize a different protein epitope on the target virus. The MBs can be incubated with a serial dilution of the cultured virus, followed by AuNP incubation. In the case of PA signal generation without the use of MBs, the MBs illustrated in this drawing would be omitted, and acoustic signal generation would occur directly from the AuNPs following laser pulse actuation.

In some embodiments of the device, AuNPs of a plurality of sizes are employed and having optical plasmonic absorption spectra specific to each size of AuNP and centered at different wavelengths, typically in the visible spectrum, with each AuNP size conjugated to a specific type of bioreceptor, such that the spectral response specific to each size of AuNP may be used to determine the target type based on spectral response specific to that size of AuNP (FIG. 7. 7). In this case, the specific target type may be recognized by the detection system as having a specific, differentiated absorption spectrum. In the simplest implementation, amplitude variation of the acoustic signal may be used to differentiate between specific targets based on their conjugated AuNP size. In an implementation with higher specificity, a plurality of lasers may be used, typically two or three, to sample the absorption spectra at different wavelengths, thereby using the relative ratio(s) of the acoustic signal to determine the specific AuNP size generating the signal, and thereby determine the target type.

Some embodiments of the device, multiplex capability may be accomplished by dividing the original specimen or sample into a plurality of aliquots after mixing with appropriate carrier solution during sample preparation, and then mixing each separate aliquot with AuNPs conjugated with different antibodies or aptamers specific to the multiple target types to be analyzed (e.g., different virus types or different protein biomarker types). The aliquots with target-specific AuNP conjugates would then be processed through the PA analysis instrument either in serial fashion, making a plurality of measurements specific to each target type, or in the case of a multichannel device, processing in parallel fashion through the instrument.

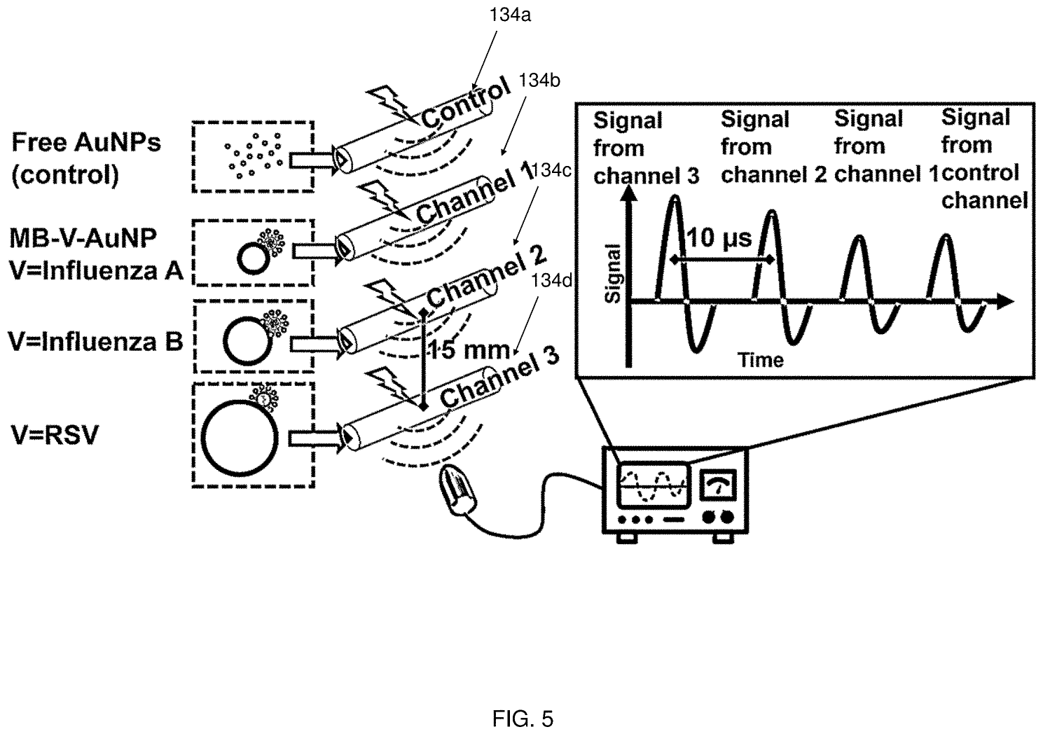

In some embodiments of the PA measurement setup, a microfluidic channel or other means will be used for sample flow. The PA signal of target-AuNP conjugates will be measured by a hydrophone (ultrasonic transducer) whose frequency covers the PA frequency of target-AuNP conjugates. A pulsed laser beam, typically focused, will create an excitation volume. The transducer will be located a select distance from the excitation spot. In one embodiment, implementing multiplex operation, and employing different size MB s, the size of MBs for a particular detected event may be inferred from the time delay between the laser pulse and the acoustic detection (FIG. 5), reflecting the spatial separation caused by passing through the DLD's pillar structure (FIG. 4A-4C).

In some embodiments, for multiplex operation employing different sizes of AuNPs, in which each particular size of AuNP is conjugated to a specific type of bioreceptor specific to a particular target (e.g., influenza viruses types A and B, SARS-CoV-2 virus, RSV, other); the amplitude of PA response to the laser pulse, or to laser pulses in the case of a plurality of lasers of different wavelengths, will enable spectroscopic identification of the AuNP size and therefore to the analyte target specific to that particular AuNP size.

In various embodiments, the selection of the laser is guided by various considerations. In implementation of the technology in an assay product, the choice of pulsed lasers, whether of a single wavelength, or with the use of a plurality of lasers of various wavelengths, will affect performance, cost, size, and power consumption. As part of the device's design, these various considerations should be balanced to optimize performance meeting market and customer requirements while minimizing cost, size, and power consumption. Based on experimental results, it is found that the pulse duration required for adequate actuation of an acoustic signal from the AuNPs may be longer than the pulse duration required to generate nanobubble cavitation, and the pulse energy required may be lower. Therefore a laser (or lasers) of lower cost and power consumption may provide adequate performance while meeting other product considerations.

Further details of various possible embodiments of the device are further disclosed below.

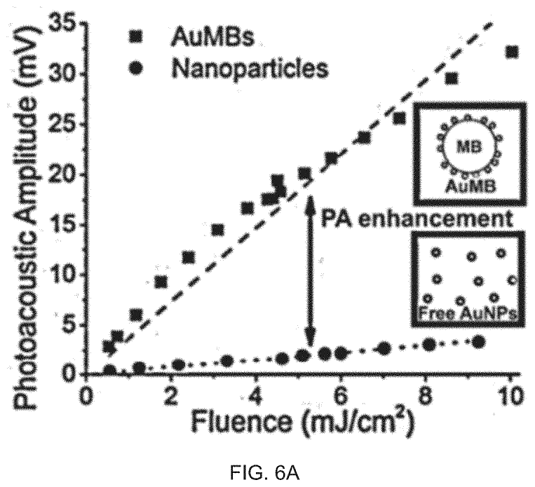

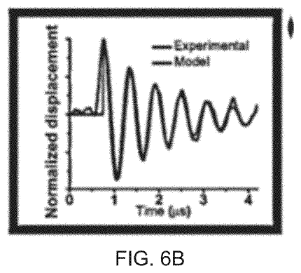

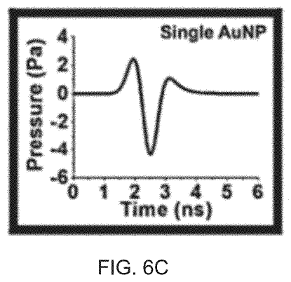

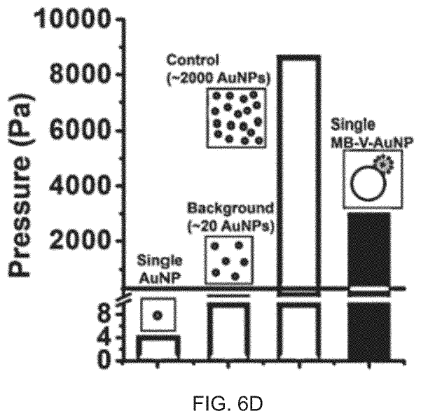

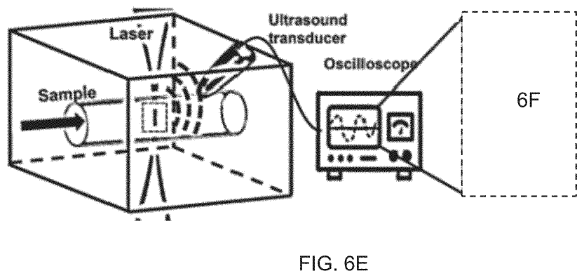

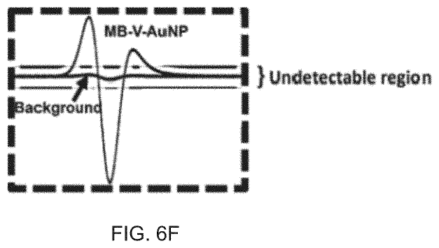

In some embodiments of the PA detection system, the microfluidic or capillary channel through which analyte solution is flowed contains a region in which a focused laser pulse occurs. In the event that this region contains one or more analyte targets, e.g., virus or protein, an acoustic signal will be generated by the plasmonic excitation of absorption modes of the AuNPs conjugated to said target. This acoustic signal may then be detected by one or more appropriately positioned acoustic transducers or hydrophones. A large number of pulses, typically which may range in the thousands to million or more range will enable digital counting of the pulses detecting an analyte target in the sample region, and thereby enable a calculation of the of the concentration of analyte targets in solution by the signal processing and data collection and analysis electronics (FIGS. 6A-6F).

For instance, in FIG. 6A, the PA amplitude of a gold nanoparticle coated microbubble (AuMB) compared with that of free gold nanoparticles (AuNPs) as familiar to those skilled in the pertinent art. The MB can convert the high frequency acoustic signal, generated by the conjugated AuNPs, into a low frequency acoustic signal, enhancing the PA signal by 8.9 times. In FIG. 6B, the vibration of an AuMB fitted to a theoretical function, as familiar to those skilled in the pertinent art. The PA generation and propagation from a single 15 nm AuNP is around 4 Pa at the position of the hydrophone. In FIG. 6C, the PA signal generated from a single AuNP at, e.g., 0.5 cm away from the particle. In FIG. 6D, there can be a comparison of PA pressure generated from single AuNP, background, control, and single MB-V-AuNP. The PA signal generated by free AuNPs is difficult to measure using a low frequency detector; only the MB-V-AuNP signal is shown to be detectable. In FIG. 6E, the microchannel setup for PA detection, including the sample, laser, ultrasound transducer, and signal processing functionality. In FIG. 6F, expected PA waveforms for different samples are illustrated. A single MB-V-AuNP can generate a signal that is an order of magnitude higher than the detection threshold of the hydrophone. In the case of PA signal generation without the use of MBs, the MBs illustrated in this diagram would be omitted, and acoustic signal generation would come directly from the AuNPs.

In some embodiments of the PA detection system, a DLD microfluidic channel (FIG. 4A-4C) contains a plurality of regions in order to separate a number of types of particles equal to the number of regions plus one: free AuNPs and the plurality of viruses of interest. In the case of three regions, for example, the MBs with diameters consisting of smallest, smaller, and small (as an example, 4, 8 and 12 μm) are collected from channels 1, 2 and 3, each capturing a previously determined virus type (as an example, Influenza virus A, B, and RSV). Free AuNPs flow into the control channel. Conjugation of both MB to virus (MB-V) and virus to AuNP (V-AuNP) is performed using antibodies as the chosen bioreceptor.

In some embodiments of the PA detection system, the DLD microfluidic channel contains a single region to separate two types of particles: free AuNPs and the virus of interest. MBs that capture, if present, the virus of interest, are collected from channel 1. Free AuNPs flow into the control channel. Conjugation of both MB to virus (MB-V) and virus to AuNP (V-AuNP) is performed using antibodies as the chosen bioreceptor.

In some embodiments of the PA detection system, system portability can be facilitated by the incorporation of small diode lasers and hydrophones for acoustic detection or by other means, thereby allowing the implementation of the device to be in a portable and/or benchtop form factor. In this instance the device can be advantageously used in PoC applications, including, but not limited to, medical practitioner offices, pharmacies, and other settings where rapid test results are desired.

In some embodiments of the PA detection system, the pillars employed for a first region (region 1) of the DLD device can triangular. In some such embodiments, compared to a circular design, the triangular pillar design provides a greater range for both pillar gap and tilt angle values and can be more suitable for small particle separation.

In some embodiments of the PA detection system, aptamers can employed in lieu of antibodies as the chosen bioreceptor for conjugation of both AuNPs. In some such embodiments, detection capability can be expanded to a large range of targets, including, but not limited to, proteins, peptides, small molecules, toxins, and live cells.

In some embodiments of the PA detection system, employing MBs, a plurality of regions and separations can be provided within the DLD device to facilitate multiplexing. In some such embodiments, depending on the number of regions and separations present, and number of different-sized MBs employed, up to a previously determined number of viruses may be differentiated in a single test.

In some embodiments of the PA detection system, the DLD device can be configured such that it incorporates an array design such as a cascade or other design. Additionally, or alternatively, the length of each region can be modified so as to increase virus separation and thereby increase test accuracy.

For any embodiments of the PA detection system disclosed herein, detection the sensitivity and specificity for analyte target detection, quantification, and differentiation can be enhanced and/or improved by the use of signal processing electronics (FIG. 6) that include microprocessor/computing components that are programed to carry out principal component analysis (PCA) on raw data, e.g., to improve the accuracy of the test results such as presented via a human interface.

For any embodiments of the PA detection system disclosed herein, data collected over many readings of the device may be compared to analysis data on the same source of samples by other means, such as PCR analysis. As a result of this analysis, machine learning algorithms may be developed that are capable of improving the sensitivity and specificity of the analysis results, and such algorithms can be incorporated into the device's data processing components.

In some embodiments of the PA detection system, the analysis throughput of the device can be enhanced by separating the sample-preparation capability from the PA measurement capability, and performing these operations separately. In some such embodiments, more than one sample preparation device may be used to queue prepared samples for analysis in the PA MB measurement portion of the device.

In some embodiments of the PA detection system, a digital assay may be performed by executing a number of sampling events on separate discrete sample volumes of analyte, with the number of sampling events being very large in comparison to the number of detection events, thereby essentially counting individual analyte targets through said detection events. By counting individual analyte targets, which may be single molecules, single protein particles, or single virions, and with the use of a large number of sampling pulses, a very high sensitivity may be achieved. Suh methods of executing digital sampling events can be accomplished by means of a pulsed laser, pulsed typically in the kilohertz range or higher, resulting in hundreds of thousands of sampling events over a period of minutes. An advantage of performing digital sampling by means of counting individual detection events is that it can provide a high degree of accuracy in quantification of the concentration of targets in the analyte solution, and avoid the need for complicated calibration procedures which would be required with analog-based measurement methods.

For any embodiments of the PA detection system disclosed herein, the system can be configured to detect viruses. Non-limiting examples include: Adenovirus, Herpes simplex, type 1, Herpes simplex, type 2, Varicella-zoster virus, Epstein-Barr virus, Human cytomegalovirus, Human herpesvirus, Human papillomavirus, BK virus, JC virus, Smallpox, Parvovirus, Rotavirus, Orbivirus, Coltivirus, Banna virus, Human astrovirus, Norwalk virus Human coronavirus 229E, Human coronavirus NL63, Human coronavirus OC43, Human coronavirus HKU1, Middle East respiratory syndrome-related coronavirus, Severe acute respiratory syndrome coronavirus, Severe acute respiratory syndrome coronavirus 2 (SARS-CoV-2), Hepatitis C virus, yellow fever virus, dengue virus, West Nile virus, TBE virus, Zika virus, Hepatitis E virus Rubella virus hepatitis A virus, poliovirus, rhinovirus, Lassa virus, Ebola virus, Marburg virus, Influenza virus Measles virus, Mumps virus, Parainfluenza virus, Respiratory syncytial virus, Rabies virus, Hepatitis D, HIV, Hepatitis B virus or combinations thereof, or other viruses familiar to those skilled in the art.

For any embodiments of the PA detection system disclosed herein, the system can be configured to detect a variety of types of protein biomarkers. Non-limiting examples include: cytokine proteins; within the cytokine category are included Interleukins, including but not limited to: major anti-inflammatory cytokines such as interleukin (IL)-1 receptor antagonist, IL-4, IL-10, IL-11, and IL-13. Leukemia inhibitory factor, interferon-alpha, IL-6, and transforming growth factor (TGF)-β, categorized as either anti-inflammatory or pro-inflammatory cytokines, under various circumstances, Lymphokines, Monokines, Interferons (IFN), colony stimulating factors (CSF), Chemokines and a variety of other proteins. The applications based on the detection of such protein biomarkers include, but are not limited to: disease diagnosis, disease prognostics, and disease therapeutics.

Applications based on the detection of virus or protein biomarkers include, but are not limited to: disease diagnosis, disease prognostics, and disease therapeutics.

One embodiment of the disclosure is a biochemical assay device 100. Referring to FIGS. 1-7 throughout, embodiments of the device can include a detection channel 130, a light source 140 and a hydrophone 150.

The detection channel 130 can be fluidly coupleable to a specimen source 110 (e.g., a container holding a specimen for analysis) to receive an analyte sample 127 that can include a biological target 112 bound to a bioreceptor gold nanoparticle conjugate 120 (AuNP-Ab). The light source 140 can be situated to send a light 142 to the detection channel, where the light includes one or more wavelengths absorbable by the bioreceptor gold nanoparticle conjugate bound to the biological target to thereby generate a photoacoustic signal 145 indicative of an individual acoustic detection event (e.g., a count) from the bioreceptor gold nanoparticle conjugates to the biological target 112 in the analyte sample. The hydrophone 150 can detect and convert a summation of the photoacoustic digital signals into an electrical signal 152.

The term photoacoustic signal as used herein means the detection of the presence or absence of a single acoustic detection event from the bioreceptor gold conjugate bound to the biological target and located within a volume of the analyte sample 127 interrogated by the light 142.

The term hydrophone as used herein means a device capable of detecting an acoustic signal and electrically communicating said signal either in an analog or digital format (e.g., an ultrasonic transducer or other ultrasound device). The hydrophone is analog detecting and that analog-detected signal is digitized in a subsequent step as disclosed in detail elsewhere herein. E.g., analog-detected signal is combined with signal processing functionality to produce a digital signal. E.g., the hydrophone receives the acoustic signal and outputs an analog signal and the analog signal processing functionality including an analog-to-digital converter, digitizes the analog-detected signal from the hydrophone and interprets the result as the presence or absence of a target in the sample volume.

In some embodiments, the bioreceptor portion of the bioreceptor gold nanoparticle conjugate can include an antibody or an apamer capable of binding to the biological target.

In some embodiments, the bioreceptor gold nanoparticle conjugate can have an average size that is a value in a range from 20 to 400 nm and thereby provide the photoacoustic signal 145 with a light absorption maximum value that is in a range of visible light from 400 to 800 nm.

Some embodiments can further including a fluid channel 105, the fluid channel including: a first inlet port 107 fluidly couplable to the specimen source 110 for holding the biological target 112 therein, and a second inlet port 115 fluidly couplable to a container 117 for holding the bioreceptor gold nanoparticle conjugate 120 (AuNP-Ab) therein, wherein a bioreceptor portion of the bioreceptor gold nanoparticle conjugate is capable of binding to the biological target.

In some such embodiments, the first inlet port 107 is fluidly couplable to a second container 155 for holding a bioreceptor microbubble conjugate 160 therein, wherein a first bioreceptor portion of the a bioreceptor microbubble conjugate 160 includes an antibody or an aptamer capable of binding to the biological target, and a second bioreceptor portion of the bioreceptor microbubble conjugate 160 includes a second antibody or a second aptamer capable of binding to the biological target.

Embodiments of the bioreceptor microbubble conjugate can include second or more antibody-functionalized microbubbles and/or second or more aptamer-functionalized microbubbles. Embodiments of the second bioreceptor portion, and more if applicable, antibody(/ies) can include streptavidin with biotin-modified antibodies that recognize a specified surface protein on the biological target.

Some such embodiments, can further include a mixing chamber 125 fluidly coupleable to the fluid channel, where a flow of the biological target and the bioreceptor gold nanoparticle conjugate from the fluid channel can be held in the mixing chamber to provide the analyte sample 127 that includes the biological target bound to the bioreceptor gold nanoparticle conjugate, and the detection channel can befluidly coupleable to the mixing chamber by a sample inlet 132 fluidly coupled to a second fluid channel 134 coupled to the mixing chamber, wherein the flow delivers the analyte sample thereto.

In some embodiments, the mixing chamber 125 can include a deterministic lateral displacement microfluidic device 400 having a microfluidic path 405 defined by microstructures 410 attached to interior walls 412 of the microfluidic channel. The microstructures can be sized and distributed to cause different velocities of the biological target bound to the bioreceptor gold nanoparticle conjugate when flowing though the microfluidic path in proportion to different-sized ones of the biological target bound to the bioreceptor gold nanoparticle conjugate.

In some such embodiments, the microstructures 405 can be arranged as an array 415 of pillars disposed in a regular pattern. E.g., the pillars can have a circular or triangular profile (e.g., cylindrical pillars or triangular prism.

In some such embodiments, the array of pillars includes different regions (e.g., Regions 1, 2, 3) in the mixing chamber, each of the regions having the pillars differently sized, or, differently spaced apart, or, disposed in different forms of the regular pattern, such that the biological target bound to the bioreceptor gold nanoparticle conjugate moves though the different regions at different velocities.

In some such embodiments, the array of pillars can include different regions (e.g., Regions 1, 2, 3) in the mixing chamber, each of the regions having the pillars differently sized, or, differently spaced apart, or, disposed in different forms of the regular pattern, such that the biological target bound to the bioreceptor gold nanoparticle conjugate moves though the different regions at different velocities.

In some embodiments, the mixing chamber can be coupled to a plurality of the second fluid channels 134a, 134b, 134c,134d (FIG. 5) where each of the second fluid channels are arranged in a pathway of the light 142 from the light source 140.

In some embodiments, the device 100 is part of an assay system 200 where the mixing chamber is part of a flow control system 205 of the assay system, and, the detection channel, the light source and the hydrophone can be part of a data acquisition system 210 of the assay system.

In some such embodiments, the assay system can further include a reservoir 162 as part of flow control system, the reservoir holding a buffered fluid 164 therein and the reservoir fluidly coupled to the mixing chamber to flow the buffered fluid to mixing chamber as the analyte sample is delivered to the detection channel.

In some such embodiments, the assay system can further include a beam splitter 165 as part of the data acquisition system, the beam splitter optically coupled to the light source 140 and wherein at least a portion of the light 142, after passing through the beam splitter, is directed through an optical fiber 167 to the detection channel. Embodiments of the assay system can further include a signal amplifier and a data conversion function 170 (e.g., an oscilloscope) as part the data acquisition system, the signal amplifier to receive the electrical signal 152 from the hydrophone and generate an amplified electrical signal 152a, and, the data conversion function to digitize the analog signal information and to generate a signal versus time profile corresponding to the amplified electrical signal corresponding to the photoacoustic signal.

In some such embodiments, the assay system can further include a user interface 215. The user interface can collect analysis information 186 about the analyte sample obtained by the data collection system 205, send an electrical control signal 188 from the computer to the flow control system to thereby control the flow of cleaning fluid through the mixing chamber, and send another electrical control signal 190 from the computer to the data acquisition system to thereby control the light from the light source.

In some embodiments, the biological target can be a virus, bacteria, pathogen, protein, metabolite, biomolecule, DNA, or RNA.

In some embodiments, the biological target can be Adenovirus, Herpes simplex, type 1, Herpes simplex, type 2, Varicella-zoster virus, Epstein-Barr virus, Human cytomegalovirus, Human herpesvirus, Human papillomavirus, BK virus, JC virus, Smallpox, Parvovirus, Rotavirus, Orbivirus, Coltivirus, Banna virus, Human astrovirus, Norwalk virus Human coronavirus 229E, Human coronavirus NL63, Human coronavirus OC43, Human coronavirus HKU1, Middle East respiratory syndrome-related coronavirus, Severe acute respiratory syndrome coronavirus, Severe acute respiratory syndrome coronavirus 2 (SARS-CoV-2), Hepatitis C virus, yellow fever virus, dengue virus, West Nile virus, TBE virus, Zika virus, Hepatitis E virus Rubella virus hepatitis A virus, poliovirus, rhinovirus, Lassa virus, Ebola virus, Marburg virus, Influenza virus Measles virus, Mumps virus, Parainfluenza virus, Respiratory syncytial virus, Rabies virus, Hepatitis D, HIV, or Hepatitis B virus.

In some embodiments the electrical signal 152 is digitized.

Those skilled in the art to which this application relates will appreciate that, based on the present disclosure, other and further combinations, additions, deletions, substitutions and modifications may be made to the described embodiments.

What is claimed is:

1. A biochemical assay device, comprising:
a detection channel fluidly coupleable to a specimen source to receive an analyte sample that can include a biological target bound to a bioreceptor gold nanoparticle conjugate;
a light source situated to send a light to the detection channel, wherein the light includes one or more wavelengths absorbable by the bioreceptor gold nanoparticle conjugate bound to the biological target to thereby generate a photoacoustic signal indicative of an individual acoustic detection event from the bioreceptor gold nanoparticle conjugates to the biological target in the analyte sample; and
a hydrophone to detect and convert a summation of the photoacoustic digital signals into an electrical signal.

2. The device of claim 1, wherein the bioreceptor portion of the bioreceptor gold nanoparticle conjugate includes an antibody or an apamer capable of binding to the biological target.

3. The device of claim 1, wherein the bioreceptor gold nanoparticle conjugate have an average size that is a value in a range from 20 to 400 nm and thereby provide the photoacoustic signal with a light absorption maximum value that is in a range of visible light from 400 to 800 nm.

4. The device of claim 1, further including a fluid channel, the fluid channel including:
a first inlet port fluidly couplable to the specimen source for holding the biological target therein, and
a second inlet port fluidly couplable to a container for holding the bioreceptor gold nanoparticle conjugate therein, wherein a bioreceptor portion of the bioreceptor gold nanoparticle conjugate is capable of binding to the biological target.

5. The device of claim 4, wherein the first inlet port is fluidly couplable to a second container for holding a bioreceptor microbubble conjugate therein, wherein a first bioreceptor portion of the a bioreceptor microbubble conjugate includes an antibody or an aptamer capable of binding to the biological target, and a second bioreceptor portion of the bioreceptor microbubble conjugate includes a second antibody or a second aptamer capable of binding to the biological target.

6. The device of claim 4, further including a mixing chamber fluidly coupleable to the fluid channel, wherein:
a flow of the biological target and the bioreceptor gold nanoparticle conjugate from the fluid channel are held in the mixing chamber to provide the analyte sample that includes the biological target bound to the bioreceptor gold nanoparticle conjugate, and
the detection channel is fluidly coupleable to the mixing chamber by a sample inlet fluidly coupled to a second fluid channel coupled to the mixing chamber, wherein the flow delivers the analyte sample thereto.

7. The device of claim 1, wherein the mixing chamber includes a deterministic lateral displacement microfluidic device having a microfluidic path defined by microstructures attached to interior walls of the microfluidic channel, wherein the microstructures are sized and distributed to cause different velocities of the biological target bound to the bioreceptor gold nanoparticle conjugate when flowing though the microfluidic path in proportion to different-sized ones of the biological target bound to the bioreceptor gold nanoparticle conjugate.

8. The device of claim 7, wherein the microstructures are arranged as an array of pillars disposed in a regular pattern.

9. The device of claim 8, wherein the array of pillars includes different regions in the mixing chamber, each of the regions having the pillars differently sized, or, differently spaced apart, or, disposed in different forms of the regular pattern, such that the biological target bound to the bioreceptor gold nanoparticle conjugate moves though the different regions at different velocities.

10. The device of claim 6, wherein the mixing chamber is coupled to a plurality of the second fluid channels wherein each of the second fluid channels are arranged in a pathway of the light from the light source.

11. The device of claim 6, wherein the device is part of an assay system wherein:
the mixing chamber is part of a flow control system of the assay system; and
the detection channel, the light source and the hydrophone are part of a data acquisition system of the assay system.

12. The device of claim 11, wherein the assay system further includes:
a reservoir as part of flow control system, the reservoir holding a buffered fluid therein and the reservoir fluidly coupled to the mixing chamber to flow the buffered fluid to mixing chamber as the analyte sample is delivered to the detection channel.

13. The device of claim 11, wherein the assay system further includes:
a beam splitter as part of the data acquisition system, the beam splitter optically coupled to the light source and wherein at least a portion of the light, after passing through the beam splitter, is directed through an optical fiber to the detection channel; and
a signal amplifier and a data conversion function as part the data acquisition system, the signal amplifier to receive the electrical signal from the hydrophone and generate an amplified electrical signal and the data conversion function to digitize the analog signal information and to generate a signal versus time profile corresponding to the amplified electrical signal corresponding to the photoacoustic signal.

14. The device of claim 11, wherein the assay system further includes:
a user interface, the user interface:
to collect source information about the biological target and send the source information to a computer of the user interface,
to collect analysis information about the analyte sample obtained by the data collection system,
to send an electrical control signal from the computer to the flow control system to thereby control the flow of cleaning fluid through the mixing chamber, and
to send another electrical control signal from the computer to the data acquisition system to thereby control the light from the light source.

15. The device of claim 1, wherein the biological target is a virus, bacteria, pathogen, protein, metabolite, biomolecule, DNA, or RNA.

16. The device of claim 1, wherein the biological target is Adenovirus, Herpes simplex, type 1, Herpes simplex, type 2, Varicella-zoster virus, Epstein-Barr virus, Human cytomegalovirus, Human herpesvirus, Human papillomavirus, BK virus, JC virus, Smallpox, Parvovirus, Rotavirus, Orbivirus, Coltivirus, Banna virus, Human astrovirus, Norwalk virus Human coronavirus 229E, Human coronavirus NL63, Human coronavirus OC43, Human coronavirus HKU1, Middle East respiratory syndrome-related coronavirus, Severe acute respiratory syndrome coronavirus, Severe acute respiratory syndrome coronavirus 2 (SARS-CoV-2), Hepatitis C virus, yellow fever virus, dengue virus, West Nile virus, TBE virus, Zika virus, Hepatitis E virus Rubella virus hepatitis A virus, poliovirus, rhinovirus, Lassa virus, Ebola virus, Marburg virus, Influenza virus Measles virus, Mumps virus, Parainfluenza virus, Respiratory syncytial virus, Rabies virus, Hepatitis D, HIV, or Hepatitis B virus.

17. The device of claim 1, wherein the electrical signal is digitized.
